(19) United States
(12) Patent Application Publication
(10) Pub. No.: US 2004/0101507 A1
(43) Pub. Date: May 27, 2004

(54) Skin cream

(76) Inventor: Janco Predovan, Manahawkin, NJ (US)

Correspondence Address: PHARMACEUTICAL PATENT ATTORNEYS, POHL & ASSOC., 55 MADISON AVENUE, 4TH FLOOR, ATTN: MARK POHL (P 4014), MORRISTOWN, NJ, 07960-6397, US

(21) Appl. No.: 10/305,571

(22) Filed: Nov. 27, 2002

(51) Int. Cl. A61K 7/06; A61K 35/78

(52) U.S. Cl. 424/74; 424/769

(57) ABSTRACT

A skin cream made from an emulsion of beeswax and olive oil, which is useful as a skin cream, a burn cream, and a wound dressing lubricant.

### BACKGROUND

Olive oil has been known for millennia, as a laxative and a cooking ingredient. Beeswax has also been known for a similar long time, and has been used for making candles, and as a machinery lubricant.

My invention entails combining beeswax and olive oil to make a skin cream. This skin cream is useful to moisten skin, as a conventional skin cream does. It has another, surprising and quite important utility; it is safe and effective as a burn cream for second and third degree burns.

### BRIEF DESCRIPTION OF THE DRAWINGS

None.

### DETAILED DESCRIPTION

I first discuss the components of the cream, and then discuss how to make my cream, and then discuss how to use my cream to treat burns.

Beeswax

Beeswax is a substance obtained from honeycombs. It is yellowish to brownish-yellow, soft to brittle consistency, with a honey-like odor and a slightly balsamic taste. It has a density of 0.95 to 0.960, and a melting point of 62-65° F. It has a saponification number of 84 and an acid number of 20. It is practically insoluble in water, is slightly soluble in cold alcohol, and is soluble in hot alcohol, in chloroform, in benzene, in ether, and in carbon disulfide.

Beeswax consists of esters of straight-chain monohydric alcohols with even-numbered carbon chains from C₂₄ to C₃₆ esterified with straight-chain acids also having even numbers of C atoms up to C₃₆ (some C₁₈ hydroxy acids). Examples of such esters are triacontanol hexadecanoate and hexacosanol hexacosanoate. These esters are mixed with about 20% (w/w) of hydrocarbons having odd-numbered straight carbon chains from C₂₁ to C₃₃. Propolis, pigments and unidentified substances amount to about six percent of the total weight of natural beeswax. The composition of beeswax is further described in the art. See, e.g., D. T. DOWNING et al., 14 AUST. J. CHEM. 253 (1961); Callow, 44 BEE WORLD 95 (1961); C. S. LETCHER, 24 KIRK-OTHMER ENCYCLOPEDIA OF CHEM. TECHNOL. 466-67 (Wiley Interscience publ., New York 3^{rd} ed. 1984).

White beeswax, also called “white wax,” “bleached yellow wax” and “bleached beeswax,” is prepared by oxidizing yellow beeswax cakes with peroxide or in sunlight. It is yellow-white in color. It has characteristics similar to those discussed above for regular beeswax, except for a slightly different taste.

The filtering process used to filter the beeswax from the honey affects the purity and color of the resulting beeswax; less stringent filtering leaves more impurities in the resulting beeswax, which, by consequence has a deeper yellow to very-light brown color. I prefer to use very light yellow Pennsylvania beeswax, which I have found to be quite pure; the very light color appears to be from sunlight or peroxide bleaching. Other waxes may be used even paraffin can be substituted for the beeswax to make the claimed cream—but such other waxes may contain undesirable organic impurities, while beeswax is natural and perhaps more pure than other waxes.

Olive Oil

Olive oil is a fixed oil obtained from ripe olives, the fruit of the cultivated olive tree Olea europaea L., Oleaceae. It is produced almost exclusively in the countries adjoining the Mediterranean Sea, Spain being the largest producer, albeit I prefer to use Croatian olive oil to make my invention. To make olive oil, whole olives are crushed in edge runner mills and the oil is expressed in open hydraulic presses. Olive oil is pale yellow to light greenish-yellow oil. It has a pleasing, delicate flavor. It becomes rancid on exposure to air. Olive oil begins to get turbid at approximately 5° to 10° F.; below 0° F., olive oil congeals into a whitish, granular mass. Olive oil has a flash point of 437° F. (225° C.), and an ignition temperature of 650° F. (343° C.), limits which should be respected in preparing my cream. Olive oil is known as slightly soluble in alcohol, and miscible with ether, with chloroform and with carbon disulfide.

Olive oil consists of mixed glycerides. A typical (albeit by no means the only) example is oleic acid (83.5%), paimic acic (9.4%), linoleic acid (4.0%), stearic acid (2.0%) and arachidic acid (0.9%). Minor constituents include squalene (up to 0.7%) and phytosterol and tocopherols (about 0.2%). The composition of olive oil is further described in the art. See, e.g., Jose M. DE SOROA Y PINEDA, EL ACEITE DE OLIVA (Dossat publ., Madrid 1944); R. F. SIMART et al., OLIVICULTURA E OLEIFICIO (Hoepli publ., Milan 1950); P. G. GAROGLIO, TECHNOLOGIA DE LOS ACEITES VEGETALES (Mendoza publ., Buenos Aires 1951); E. W. ECKEY, VEGETABLE FATS AND OILS, (Reinhold publ., New York 1954).

I prefer to use olive oil from Croatia. This is because olive groves in Croatia do not overuse pesticides nor chemical fertilizers (this is because, as a former communist country, Croatia has not had the foreign exchange reserves to purchase large amounts of pesticides and chemical fertilizers).

Making the Cream

Given these components, one can make them into a skin cream. I prefer to make the cream using the following ratio of ingredients:

| Ingredient | Quantity |
|---|---|
| Olive oil (pure Croatian) | 500 ml |
| Bees wax (bleached) | 125 grams |
| Water | q.v. |

The precise oil:wax ratio can be varied; for 500 ml of oil, for example, one may use 100 grams of beeswax, to make a less viscous cream.

To make the cream, combine the olive oil and the beeswax in a clean, covered heating container. Heat the container, stirring occasionally until the beeswax melts. The may be heated at a fairly low temperature, just over the beeswax melting point (for example, approximately 100° F.), until the beeswax is melted.

Once the beeswax melts completely, heat the mixture until small bubbles appear on the bottom of the heating container and the mixture is on the verge of boiling. The mixture may smoke faintly. This will be near or at the mixture's flash point (the flash point of oil is 437° F. (225° C.)). Heating the mixture to the boiling point makes the mixture so hot it can “flash”—catch fire explosively. Thus, this heating should be done in a protective hood or other flame-proof and non-explosive conditions.

Furthermore, as soon as small bubbles appear on the bottom of the heating vessel, immediately remove the heating vessel from the heat source, to prevent a flash fire. Then, pour the hot mixture into cool water. The water must be cool enough, and in enough volume, to congeal the mixture. T have found that 10-15 liters of water, at about 41-68° F. (5-20° C.), works well. Note that the hot mixture is added to water, rather than visa versa. Adding cool water to boiling oil would make the water evaporate and vaporize explosively, splattering hot oil.

The water cools and congeal the beeswax—olive oil mixture, which will, when poured into the water, first sink to the bottom of the water and then thicken and float to the top of the water.

I refer to this congealed cream an “emulsion” for lack of a better word. I have ho reason to believe that the precise physical form of the mixture is important; I would expect a colloidal suspension, for example, or a matrix to be equivalent.

Skim the congealed oil-wax cream from the top of the water. Excess water should be removed from the cream by compressing the congealed cream. The cream will be quite viscous and sticky. Return the cream to a clean heating container.

In the heating container, heat the oil-wax cream until it is again liquid and at the boiling point, immediately remove from heat, and add it to a fresh quantity of cool water to congeal it. I prefer to repeat this process of melting and congealing five to ten times; so repeating it may help to wash from the oil and the beeswax soluble contaminants.

After the final congealing, the congealed cream is collected from the top of the water, excess water squeezed out, and then the cream is decanted into containers. The resulting cream, so packed in jars or tubes, may be stored under cool, refrigerated conditions to prevent the emulsion from melting and separating. So stored, the cream appears stabile against degradation. This is interesting, as it is conventionally known that natural olive oil oxidizes and becomes rancid fairly rapidly. The ability of my cream to last may be due to an anti-oxidant chemical component in beeswax, or to the physical form of the cream (an emulsion, which might become rancid on the thin surface exposed to air, but which emulsion may physically block atmospheric oxygen gas from physically penetrating below this surface into the cream).

Using the Cream

My cream is a quite good product for general skin care. It is all natural, in contrast to many conventional skin creams.

Unlike conventional skin creams, my skin cream is safe and extremely effective cream for use on burns. It has been tested in hospitals on severe-burn patients. It has been found to hasten the growth of new skin and prevent scarring on healing skin.

Hastening the growth of new skin may be due to the emollient action of olive oil penetrating the skin, supplementing or replacing the skin's naturally-occurring lipid component.

Alternatively, this may be due to the oil-wax compound creating a physical vapor barrier. Severe skin burns may damage the skin's physical vapor barrier which keeps the skin tissue's moisture in the skin tissue, and may damage the sweat glands present in healthy skin, reducing the skin's ability to moisturize itself. My cream may act as a replacement vapor barrier, sealing in the skin's natural moisture, preventing the skin from drying out, and hasting the growth of healthy replacement tissue.

This cream also prevents opportunistic infections which often accompany severe burns. In addition to hastening the growth of replacement skin—and reducing the incidence of scar tissue in the replacement skin—my cream reduces the incidence of opportunistic infection in severe burns. This property may be due to the cream acting as a vapor barrier, preventing the growth of aerobic bacteria. As my cream appears to also halt the growth of septic or anaerobic bacterial infection, it appears that a vapor barrier mechanism is not the only anti-bacterial mechanism. Perhaps an antibacterial compound present in beeswax creates this function. This does not seem far-fetched, as anyone who has found a years-old jar of honey in the back of their pantry knows that honey is not particularly vulnerable to rotting from fungal nor bacterial decay, and honey thus very well may contain some natural anti-fungal or anti-bacterial agent.

Furthermore, as a burn and wound cream, the cream prevents the wound dressing from sticking to the wounded skin. This enables the dressing to be changed frequently, without injuring the healing skin tissue. This may be due to the cream acting as a lubricant on the skin, or on the dressing itself. Alternatively, this may be due to the cream creating a physical barrier layer between the injured tissue and the wound dressing.

As a burn cream for 2^{nd} and 3^{rd} degree burns, the cream is applied once a day for three weeks. When so used, the injury must first be debreeded of necrotic tissue. This process is known in the art of dermatology. The cream is applied to the wound dressing. The dressing is then placed on the wound with the cream contacting the wound, covering the burned area completely. The results are astonishing, as new skin grows back without any visible scars after about three weeks.

Conclusion

It will become apparent to one of skill in the art that my invention may be modified to create products which vary from the examples discussed herein, but still fall within the spirit and scope of my invention as claimed. For example, the ratios of beeswax to olive oil may be varied, as may the number of mixing cycles, the mixing temperature, and the like. Thus, while I discuss my preferred embodiment in detail, I intend the legal coverage of my patent to be defined not by the specific examples I discuss, but by the claims appended here.

I claim:

1. A skin cream comprising an emulsion of olive oil and beeswax.

2. The skin cream of claim 1, said olive oil and said beeswax present in a ratio of about 0.5 liters of olive oil:125 grams of beeswax.

3. The skin cream of claim 1, made by the process of:
mixing melted beeswax and olive oil together to form a mixture; and
cooling said melted beeswax and olive oil mixture to form a cream.

4. The skin cream of claim 3, said cooling step performed by adding said melted beeswax and olive oil mixture to cool water.

5. A method for treating a skin wound, comprising:
topically applying the cream of claim 1 to said wound for an effective period of time.

6. The method of claim 5, wherein said wound is a burn.

7. A method for preventing adhesion of a wound dressing to a wound, comprising applying the cream of claim 1 to said wound dressing.

8. In a skin cream, the improvement comprising an emulsion of beeswax and olive oil.

9. An article of manufacture comprising: a wound dressing, and the cream of claim 1 applied to said wound dressing.

10. A method for maintaining healthy skin or promoting the growth of healthy skin, said method comprising topically applying the cream of claim 1.
